(19) United States
(12) Patent Application Publication
(10) Pub. No.: US 2022/0265978 A1
(43) Pub. Date: Aug. 25, 2022

(54) APPLICATOR AND ASSEMBLY WITH PIERCING FEATURE

(71) Applicant: EPIC BRANDS, LLC, Santa Monica, CA (US)

(72) Inventor: Jana Bobosikova, Santa Monica, CA (US)

(21) Appl. No.: 17/182,089

(22) Filed: Feb. 22, 2021

Related U.S. Application Data
(63) Continuation of application No. 17/182,071, filed on Feb. 22, 2021.

(51) Int. Cl. A61M 35/00; A61M 5/28; A45D 34/04; A45D 40/26

(52) U.S. Cl. CPC A61M 35/003; A61M 5/286; A45D 34/04; A45D 40/26

(57) ABSTRACT

The present invention relates to the field of flexible containers, specifically portable containers that hold solid or liquid cosmetics or personal care products. Embodiments of the invention comprise a metal flexible container with a removable metal applicator and a removable metal cap. Said applicator may be affixed to the container by threads or other means. Said cap may be affixed to the container or to the applicator by threads or by other means. Said applicator may be shaped in order to feel comfortable against a user's skin or lips. Said cap may comprise a feature or features that allow for convenient portability of the assembled cap, applicator, and container. Said cap may also comprise a feature or features that allow the assembled cap, applicator, and container to be attached to bathroom fixtures affixable to a keychain so that a user may conveniently carry the cosmetic container on their person.

### BACKGROUND OF INVENTION

In the field of cosmetics and personal care consumer products, flexible containers exist to hold liquid products so that a user may carry said products with them and apply said products whenever desired. Many of said containers comprise applicators, which are devices permanently or removably attached to the containers that aid in dispensing the products and applying the products to the users' skin, lips, hair, etc. Many of said containers further comprise removable caps to prevent the liquids from exiting the containers when not desired by the users.

Most of said containers that exist in the art are made of non-recyclable and/or non-biodegradable materials, and thus are wasteful and potentially harmful to the environment. Since these containers are portable in size, they cannot hold as much product as a larger container and thus are thrown away more often.

The applicators and caps of said containers are also frequently non-recyclable and/or non-biodegradable. Furthermore, the applicators are usually permanently attached to the containers, and thus are thrown away with the containers. This combination of materials, inherited from historical tooling specialization in the container industry and related costs (i.e. 1 material in container, another in closure/cap or dispenser) adds even more, often non-recyclable waste caused by flexible containers.

Some flexible containers are made entirely of recyclable metal. However, the applicators of these metal containers are not designed for optimum application of product to a user. Many of the applicators and orifices, of metal tubes particularly, are uncomfortable on the users' skin or lips and prevent wider-spread use of sustainable materials in the cosmetic categories. Furthermore, when metal containers are in use, they are affixed with plastic caps, so while the metal container may be recyclable, the plastic cap is not.

There exists a need for a flexible container made of recyclable metal that comprises a removable applicator and a removable cap, such that a user may store said applicator and cap to be used with other cosmetic containers, and thus reduce waste. There further exists a need that said applicator be comfortable against a user's skin or lips in order for these products to be adopted by the customers.

An option to have a user-friendly applicator in the same material as the container affixed, allows complete recyclability and re-circulation of the material (e.g. 75% of aluminum ever extruded is still in circulation) as well as long-term reduction in use of materials, as those applicators and caps may be keep-sake items, and only the containers with product may be re-purchased/manufactured.

### SUMMARY OF INVENTION

The present invention relates to the field of flexible containers, specifically portable containers that hold liquid cosmetics or personal care products. Embodiments of the invention comprise a metal flexible container with a removable metal applicator and a removable metal cap. Said applicator may be affixed to the container by threads or other means. Said cap may be affixed to the container or to the applicator by threads or by other means. Said applicator may be shaped in order to feel comfortable against a user's skin or lips. Said cap may comprise a feature or features that allow for convenient portability of the assembled cap, applicator, and container. Said cap may also comprise a feature or features that allow the assembled cap, applicator, and container to be attached to bathroom fixtures.

### BRIEF DESCRIPTION OF DRAWINGS

FIG. 1 is an assembled view of a flexible container and removable cap.

FIG. 2 is an expanded view of a flexible container, removable applicator, and removable cap.

FIG. 3 is a cross-section view of an assembled flexible container, threaded applicator, and threaded cap.

FIG. 4 is a cross-section view of a threaded applicator.

FIG. 5 is a sectional view of FIG. 5 that better illustrates a piercing feature on the threaded applicator.

FIG. 6 is a cross-section view of a removable cap with a keychain attachment.

FIG. 7 is a close-up cross-section view of an assembled flexible container, threaded applicator, and threaded cap.

### DETAILED DESCRIPTION

The description of the invention provided herein is for exemplary purposes and is not intended to limit the invention to any of the embodiments described herein. The figures used to support this specification are not intended to limit the invention to any size, shape, presence of any specific features, or any specific physical or mechanical properties. The claimed invention is best understood by the appended claims.

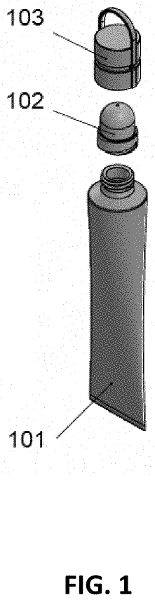

FIG. 1 illustrates an assembly comprising a flexible container 101, an applicator 102, and a cap 103. The cap and applicator may be removable and re-attachable to the flexible container. In some embodiments, the cap may be removable and re-attachable to only the applicator and not to the flexible container. The flexible container, applicator, and cap may all be hollow and may all comprise interior and exterior walls. The flexible container may contain a liquid. Said liquid may be a cosmetic or personal care product. The purpose of the cap may be to prevent the liquid contained within the flexible container from exiting the flexible container when undesired by a user. The purpose of the applicator may be to apply the liquid to a user's skin, lips, hair, etc.

In some embodiments, the applicator may comprise an open end and a closed end. The open end of the applicator may comprise internal threads that engage with corresponding external threads on the flexible container in order to attach the applicator to the flexible container. The closed end of the applicator may comprise an orifice so that liquids may pass into the open end of the applicator from the container, pass through the applicator, and pass out of the closed end of the applicator via the orifice.

In some embodiments, the cap may comprise an open end and a closed end. The open end of the cap may comprise external threads that engage with corresponding internal threads on the applicator in order to attach the cap to the applicator. The closed end of the cap may be completely closed as to not allow liquid to exit the assembly when the cap is attached to the assembly.

The flexible container may comprise an open end and a closed end. The open end of the flexible container is the end that may comprise external threads and is the end that engages with the open end of the applicator to allow the passage of liquids between the flexible container and the applicator. The open end of the flexible container may comprise a membrane that seals the open end of the flexible container until said membrane is removed or pierced.

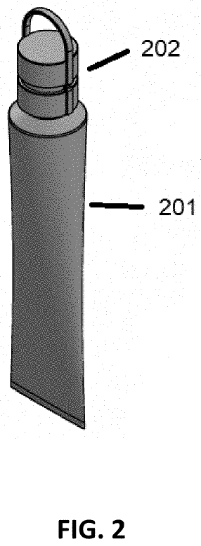

FIG. 2 illustrates the assembly of FIG. 1 where all of the features are fully assembled. In FIG. 2, the applicator is not visible because it is between the flexible container 201 and the cap 202. It should be noted that while the attachment of the components described herein may be threads, said attachment may also be any other means of attachment, such as but not limited to magnets, taper lock, Luer-type lock, and snap features.

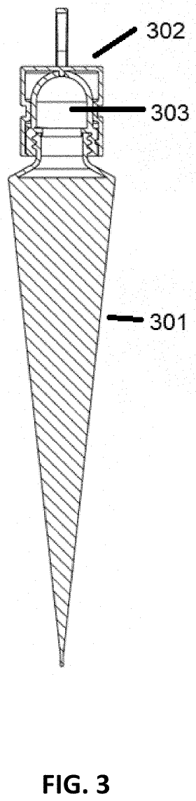

FIG. 3 illustrates a cross-section view of the assembly comprising the flexible container 301, cap 302, and applicator 303 as they exist in some embodiments of the invention. In FIG. 3, the applicator is threaded onto the flexible container by means of external threads on the open end of the applicator and external threads on the flexible container. The cap is threaded onto the applicator by means of internal threads on the open end of the cap and external threads on the applicator.

The internal and external threads on flexible container, applicator, and cap may be any shape or size. In some embodiments, the threads that join the cap and applicator are spherical threads.

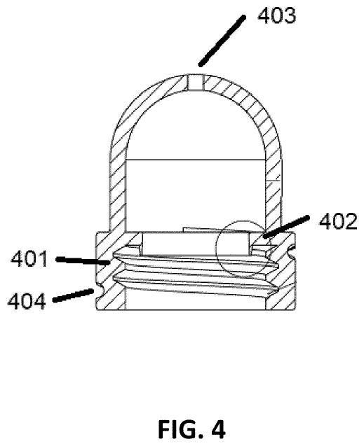

FIG. 4 illustrates a cross-section view of the applicator as it exists in some embodiments of the invention. Shading in FIG. 4 is used to represent material. Lack of shading in FIG. 4 is used to represent open space or hollow space. The applicator may comprise an open end and a closed end. The applicator may comprise internal threads 401 that engage with the external threads of the flexible container. The applicator may comprise internal spherical threads 404 that engage with the external threads of the cap. The internal threads 401 and/or the internal spherical threads 404 may be located at the open end of the applicator.

The applicator may comprise an orifice 403 at the closed end of the applicator to allow liquids to pass from the flexible container, through the open end of the applicator, through the applicator, and out of the orifice in the closed end of the applicator. The closed end of the applicator may be a rounded or domed shape to be more comfortable on a user's skin or lips when using the assembly to apply liquids to the user's skin, lips, hair, etc.

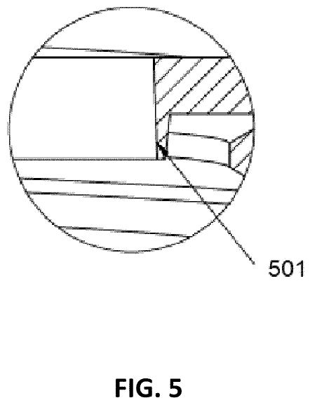

The applicator may comprise a piercing feature 402 that pierces the membrane of the flexible container when the applicator is connected to the open end of the flexible container. FIG. 5 illustrates a section view of the piercing feature 501. The piercing feature may be located on the interior wall of the applicator and may be situated between the internal threads 401 and the closed end of the applicator, so that when the internal threads of the applicator engage with the external threads of the flexible container, the piercing feature contacts the membrane of the flexible container and pierces the membrane to allow liquids to flow from the flexible container to the applicator. Due to its location as part of the internal wall of the applicator between the internal threads 401 and the closed end of the applicator, the piercing feature may serve as an entrance to a hollow passageway in which liquids flow upon exiting the flexible container into the applicator.

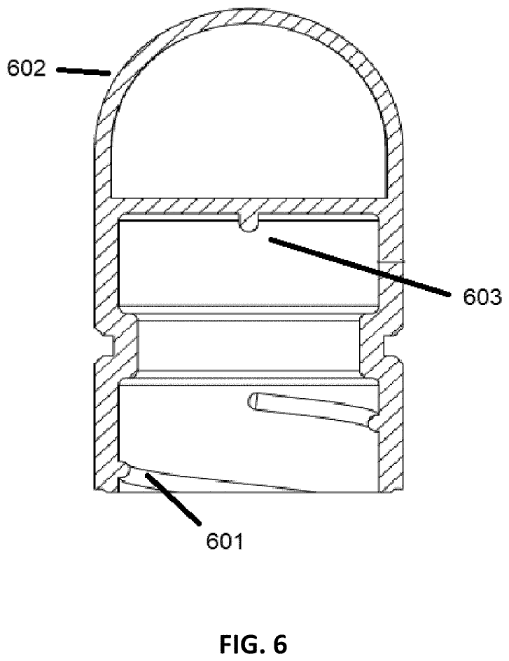

FIG. 6 illustrates a cross-section view of the cap as it exists in some embodiments of the invention. Shading in FIG. 6 is used to represent material. Lack of shading in FIG. 6 is used to represent open space or hollow space. The internal threads 601 at the open end of the cap are illustrated as spherical threads. The closed end of the cap may comprise a keychain feature 602 that may serve to allow the assembly to be attached to a keychain, carabiner, clip, or the like. The keychain feature may be any shape, size, or material. In some embodiments of the invention, the keychain feature comprises an arc of material permanently attached to the closed end of the cap to allow for objects such as keychain rings or carabiners to pass over and under the arc. The keychain feature illustrated in FIG. 6 is present in only some embodiments of the invention. Other embodiments of the invention comprise a feature or features in a similar location on the cap. Said feature or features may be used to allow the closed end of the cap to act as a base in order for the assembly to be free-standing. Other uses of said feature or features may be to attach the assembly to various bathroom fixtures.

In some embodiments of the invention, the cap comprises a nub 603 that fits inside of the orifice in the closed end of the applicator when the cap is attached to the applicator. The nub creates a seal that prevents liquids from exiting the assembly when the cap is attached to the assembly via the applicator.

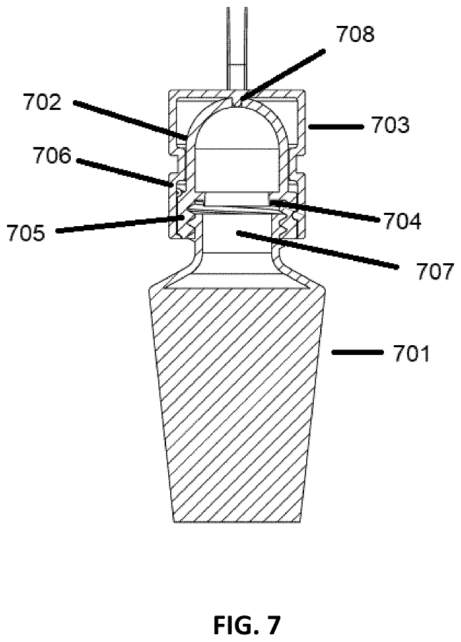

FIG. 7 illustrates a cross-section view of the assembly with the flexible container 701, the applicator 702, and the cap 703. The applicator is engaged with the flexible container so that the piercing feature 704 has pierced the membrane that may be present at the open end of the flexible container. The threads 705 of the applicator and of the flexible container have been engaged in order to connect the applicator to the flexible container. The spherical threads 706 of the applicator and of the cap have been engaged in order to connect the cap to the applicator. Since the cap is attached to the applicator, the nub 708 fits into the orifice at the open end of the applicator in order to create a seal to prevent liquids from exiting the assembly.

As illustrated in FIG. 7, when the applicator and flexible container are attached to one another, a hollow passageway 707 is formed that allows liquids to flow from the flexible container to the applicator so that said liquids may be applied to a user's skin or lips.

It should be noted that while the figures described herein depict a tube-like container, the flexible container is not limited to any particular size or shape. The flexible container may be a tube, bag, bottle, box, or any other form of flexible packaging for liquids.

What is claimed is:

1. An applicator for applying cosmetics or personal care products (herein “products”) to a user, comprising:
an open end;
a closed end; and
a piercing feature,
wherein when the open end of the applicator is removably attached to an open end of a flexible container, the piercing feature pierces a membrane located at the open end of the flexible container to allow products to pass from the flexible container to the applicator.

2. The applicator of claim 1, wherein the applicator is removably attached to the open end of the flexible container by a first set of internal threads on the applicator and a set of external threads on the open end of the flexible container.

3. The applicator of claim 2, further comprising a second set of internal threads, wherein the second set of internal threads engage with a set of external threads at an open end of a cap to removably attach the cap to the applicator.

4. The applicator of claim 1, wherein the closed end of the applicator is rounded or domed in shape.

5. The applicator of claim 1, wherein the closed end comprises an orifice.

6. The applicator of claim 1, wherein the applicator is made of metal.

7. The applicator of claim 6, wherein the applicator is made at least partly of aluminum.

8. An assembly, comprising:
a flexible container containing a product and comprising an open end, a closed end, and a membrane;
an applicator comprising an open end, a closed end, and a piercing feature; and
a cap comprising an open end and a closed end,
wherein when the open end of the applicator is removably attached to the open end of the flexible container, the piercing feature pierces the membrane to allow the product to pass from the flexible container to the applicator.

9. The assembly of claim 8, wherein the applicator is removably attached to the open end of the flexible container by a first set of internal threads on the applicator and a set of external threads on the open end of the flexible container.

10. The assembly of claim 9, wherein the applicator further comprises a second set of internal threads, wherein the second set of internal threads engage with a set of external threads at the open end of a cap to removably attach the cap to the applicator.

11. The assembly of claim 10, wherein the second set of internal threads of the applicator and the set of external threads of the cap are spherical threads.

12. The assembly of claim 8, wherein the closed end of the applicator is rounded or domed in shape.

13. The assembly of claim 8, wherein the flexible container, applicator, and cap are made of metal.

14. The assembly of claim 13, wherein the flexible container, applicator, and cap are made at least partly of aluminum.

15. The assembly of claim 8, wherein the cap further comprises a keychain feature to facilitate an attachment of the cap to another device, wherein said attachment allows for convenient transportation of the assembly when the cap, applicator, and flexible container are attached to one another.

16. The assembly of claim 8, wherein the cap further comprises a nub that fits inside of an orifice at the closed end of the applicator when the cap is attached to the applicator.

17. The assembly of claim 16, wherein when the cap is attached to the applicator, the liquid may flow from the flexible container to the applicator but may not flow out of the orifice at the closed end of the applicator.

18. The assembly of claim 8, wherein when the applicator is attached to the flexible container and wherein the cap is not attached to the applicator, the liquid may flow from the flexible container through the applicator and out of an orifice at the closed end of the applicator.
